(19) United States
(12) Patent Application Publication
(10) Pub. No.: US 2010/0210847 A1
(43) Pub. Date: Aug. 19, 2010

(54) PROCESS FOR THE PREPARATION OF PANTOPRAZOLE SODIUM AND PANTOPRAZOLE SODIUM SESQUIHYDRATE

(75) Inventors: Ashish M. Trivedi, Gujarat (IN); Shailendra Kumar Singh, Haryana (IN); Neera Tewari, Haryana (IN); Mohan Prasad, Haryana (IN)

Correspondence Address: Ranbaxy Inc., Intellectual Property Department, 600 College Road East, PRINCETON, NJ, 08540, US

(73) Assignee: RANBAXY LABORATORIES LIMITED, New Delhi (IN)

(21) Appl. No.: 12/669,553

(22) PCT Filed: Jul. 17, 2008
(86) PCT No.: PCT/IB2008/052886

§ 371 Date: Apr. 20, 2010

(30) Foreign Application Priority Data
Jul. 17, 2007 (IN) 1506/DEL/2007

(51) Int. Cl. C07D 401/12

(52) U.S. Cl. 546/273.7

(57) ABSTRACT

The present invention relates to a process for the preparation of pantoprazol sodium sesquihydrate of formula (I) and pantoprazole sodium.

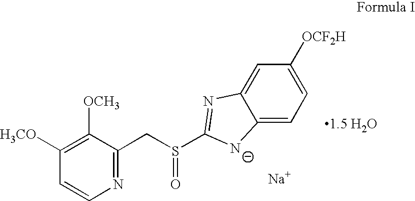

### FIELD OF THE INVENTION

The present invention relates to processes for the preparation of pantoprazole sodium sesquihydrate and pantoprazole sodium.

### BACKGROUND OF THE INVENTION

Pantoprazole sodium sesquihydrate is chemically, sodium 5-(difluoromethoxy)-2-[[3,4-dimethoxy-2-pyridinyl)methyl]sulfinyl]-1H-benzimidazole sesquihydrate and is represented by Formula I

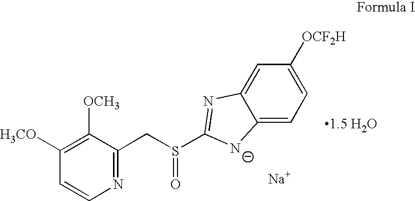

It is known from U.S. Pat. No. 4,758,579 and is used as an inhibitor of acid gastric secretion for the treatment of gastric ulcer, for the short-term treatment of erosive esophagitis associated with gastroesophageal reflux disease (GERD), maintenance of healing of erosive esophagitis and pathological hypersecretory conditions including Zollinger-Ellison syndrome.

Several processes for the preparation of pantoprazole sodium sesquihydrate are known in literature such as those described in U.S. Pat. Nos. 7,081,534 and 7,060,839, U.S. Publication No. 2004/0177804, PCT Publication No. WO 2007/017890 and J. Med. Chem., (1992), 35(6), 1049, which are herein incorporated by reference.

U.S. Pat. No. 7,081,534 describes a process for the preparation of pantoprazole sodium sesquihydrate comprising methoxylation of a compound of Formula II,

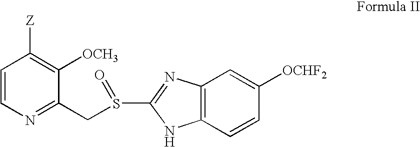

wherein Z is a leaving group such as halogen or —OH group, with sodium methoxide in methanol at elevated temperature, extraction of pantoprazole sodium salt with methyl ethyl ketone and acetone followed by purification. The process suffers from the drawback that methoxylation of the intermediate of Formula II may lead to formation of undesired side-product, formed by the methoxylation at nitrogen linked to hydrogen of the benzimidazole ring, due to which additional chromatographic purification steps are needed and the yields obtained are low.

U.S. Pat. No. 7,060,839 describes a process for the preparation of pantoprazole sodium sesquihydrate comprising selective methoxylation of a compound of Formula II with a methoxylating agent in an aprotic polar solvent to obtain crude product followed by purification. The process involves the use of costly solvents, such as N,N-dimethylformamide or N,N-dimethylacetamide, and higher temperatures for subsequent removal of the solvent at the end of the reaction, which is not suitable on an industrial scale.

U.S. Publication No. 2004/0177804 describes processes for the preparation of pantoprazole sodium sesquihydrate comprising forming a solution of pantoprazole and sodium hydroxide in a diluent, overnight stirring followed by addition of an anti-solvent to obtain pantoprazole sodium sesquihydrate. It also describes the preparation of pantoprazole sodium sesquihydrate by forming a heterogeneous mixture obtained by contacting pantoprazole sodium and a diluent, and recovering pantoprazole sodium sesquihydrate from the heterogeneous mixture. The processes are not suitable for industrial scale synthesis as they either involve the use of long reaction times, such as overnight stirring and use of an additional solvent, as an anti-solvent, for facilitating the crystallization, thus adding to the cost of the reaction or involve heterogeneous mixing of the reactant and the solvent due to which the reaction may not be complete and the product may contain un-reacted pantoprazole sodium, thus leading to poor yield and purity of the final product.

WO 2007/017890 describes a process for the preparation of pantoprazole sodium sesquihydrate comprising forming a suspension of pantoprazole sodium in a solvent mixture comprising ether and water followed by isolation. The process involves the preparation of pantoprazole sodium sesquihydrate using a two-phase system due to which the reaction may not be complete which may affect the yield and purity of the product and additional purification steps need to be carried out for obtaining product of better purity.

J. Med. Chem., (1992), 35(6), 1049 describes a process for the preparation of pantoprazole sodium sesquihydrate by drop-wise addition of sodium hydroxide to a solution of of pantoprazole free base in a mixture of ethanol and dichloromethane followed by addition of diisopropyl ether, as an anti-solvent, to obtain the product. The process involves the use of an additional solvent, as an anti-solvent, for carrying the reaction, which adds to the cost of the process and is also not recommended for an industrial scale preparation. Also, the process involves the use of ethanol in excess due to which isolation of the product is difficult and the yield is low.

U.S. Publication No. 2005/075370 describes a process for the preparation of pantoprazole sodium using sodium hypochlorite as an oxidizing agent in the oxidation step followed by addition of an anti-solvent. Although the process overcomes the problem of over-oxidation by limiting the formation of sulphone impurity of Formula III

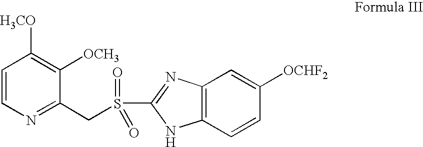

which other-wise is difficult to eliminate, by known purification methods such as recrystallization due to the formation of mixed crystals with sulphoxide, it involves the use of an additional solvent, as an anti-solvent, for isolation, which adds to the cost of the process.

WO 2006/064249 describes a process for the preparation of pantoprazole sodium comprising the reaction of 2-mercapto-5-difluoromethoxy benzimidazole with 2-chloromethyl-3,4-dimethoxypyridine hydrochloride in the presence of a base and a phase-transfer catalyst followed by treatment of the sulphide intermediate with aqueous sodium hypohalite solution.

WO 2007/026188 describes a process for the preparation of pantoprazole sodium using sodium hypochlorite, in the presence of a phase-transfer catalyst, as an oxidizing agent.

Due to the drawbacks associated with the processes known in the literature for the preparation of pantoprazole sodium and pantoprazole sodium sesquihydrate, there is a need for the development of industrially advantageous, cost effective, less time-consuming processes for the preparation of pantoprazole sodium and pantoprazole sodium sesquihydrate which overcome the problem associated with over-oxidation of the sulphide intermediate, without using a phase-transfer catalyst, and leads to easier isolation of pantoprazole sodium sesquihydrate of high purity and better yield.

The present inventors have developed industrially advantageous processes for the preparation of pantoprazole sodium and pantoprazole sodium sesquihydrate of high purity and better yield which circumvent the drawbacks associated with the processes known in the prior art.

### SUMMARY OF THE INVENTION

In a first aspect, the invention provides a process for the preparation of pantoprazole sodium sesquihydrate of Formula I

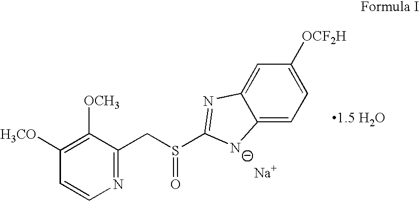

comprising contacting pantoprazole sodium of Formula IV

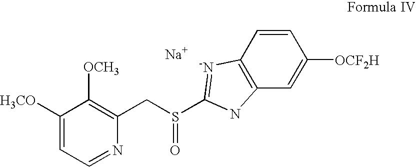

with a mixture of chlorinated solvent and alcohol, optionally seeding the reaction mixture, followed by isolation.

A second aspect of the invention provides a one-pot process for the preparation of pantoprazole sodium of Formula IV

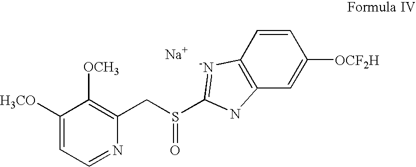

comprising condensation of 2-mercapto-5-difluoromethoxy benzimidazole of Formula V

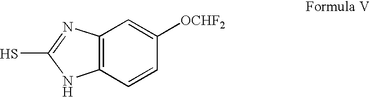

with 2-chloromethyl-3,4-dimethoxyptridine hydrochloride of Formula VI,

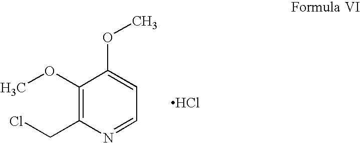

to obtain a sulphide intermediate of Formula VII,

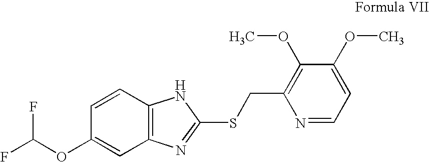

adding an oxidizing agent to obtain pantoprazole free base of Formula VIII

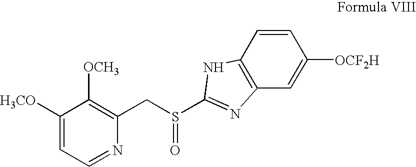

followed by its conversion to pantoprazole sodium, wherein the condensation and oxidation reactions are carried out in the absence of a phase transfer catalyst.

A third aspect of the invention provides pantoprazole sodium, obtained by the process of the present invention, substantially free of sulphone impurity.

A fourth aspect of the invention provides pantoprazole sodium sesquihydrate, obtained by the process of the present invention, substantially free of sulphone impurity.

### DETAILED DESCRIPTION OF THE INVENTION

Pantoprazole sodium, used as a starting material for the preparation of pantoprazole sodium sesquihydrate in the first aspect of the invention, can be obtained by any of the processes described in the literature such as those described in U.S. Pat. Nos. 4,758,579; 4,508,905; 4,628,098; 5,045,552; 7,081,534; and 7,060,839, U.S. Publication No. 2004/0177804, PCT Publication Nos. WO 91/19710; WO 01/ 68594; WO 2006/049486; WO 2006/064249; WO 2007/017890; and WO 2007/026188, and J. Med. Chem., (1992), 35(6), 1049, which are herein incorporated by reference only.

Pantoprazole sodium, used as a starting material for the preparation of pantoprazole sodium sesquihydrate in the first aspect of the invention, can also be obtained by the methods described in the second and third aspect of the present invention.

Pantoprazole sodium, used as an intermediate for the preparation of pantoprazole sodium sesquihydrate, may be used as a solution directly from a reaction mixture in which it is formed and may be used as such without isolation.

The term “contacting” includes dissolving, slurrying, stirring or a combination thereof.

The chlorinated solvent, used for the preparation of pantoprazole sodium sesquihydrate, may be selected from the group comprising of chloroform, dichloromethane, dichloroethane and the like. Preferably, the chlorinated solvent used is dichloromethane.

The alcohols, used for the preparation of pantoprazole sodium sesquihydrate, may be selected from the group comprising of straight and branched chain alcohols such as methanol, ethanol, n-propanol, iso-propanol and the like, cyclic alcohols such as cyclopentanol, cyclohexanol and the like, aromatic alcohols such as substituted or un-substituted benzyl alcohols and the like. Preferably, the alcohol used is ethanol.

The conversion of pantoprazole sodium to pantoprazole sodium sesquihydrate may be facilitated by adding a seed crystal to the reaction mixture. The reaction mixture may be cooled to a temperature of about −10 to about +10° C. Preferably, the reaction mixture is cooled to a temperature of about 0-5° C. Seed may be prepared by the method described in example 3 of this application.

Isolation of pantoprazole sodium sesquihydrate may be accomplished by concentration, precipitation, cooling, filtration or centrifugation or a combination thereof followed by drying. Preferably, isolation is achieved by precipitation.

The intermediates, 2-mercapto-5-difluoromethoxy benzimidazole of Formula V and 2-chloromethyl-3,4-dimethoxypyridine hydrochloride of Formula VI, to be used as starting materials for the preparation of pantoprazole sodium of Formula IV in the second and third aspect of the invention, may be obtained by any of the processes described in the literature such as those described in U.S. Pat. No. 6,723,852, PCT Publication Nos. WO 2006/064249; WO 02/28852; and WO 2004/092142, and Spanish Patent Nos. ES 2036948, ES 0174726, ES 2036502, ES 2060541 and ES 2036948, which are herein incorporated by reference only. The intermediates of Formula V and Formula VI may be obtained as a solution directly from a reaction mixture in which it is formed and may be used as such without isolation.

Condensation of 2-mercapto-5-difluoromethoxy benzimidazole of Formula V with 2-chloromethyl-3,4-dimethoxypyridine hydrochloride of Formula VI may be carried out by addition of 2-chloromethyl-3,4-dimethoxypyridine hydrochloride to an aqueous solution of 2-mercapto-5-difluoromethoxy benzimidazole in the presence of a base.

The base to be used for the condensation reaction may be selected from the group comprising of inorganic bases such as hydroxides, carbonates, bicarbonates, acetates, alkoxides of alkali and alkaline earth metals. Examples of inorganic bases may include sodium hydroxide, potassium hydroxide, sodium carbonate, potassium carbonate, potassium bicarbonate and the like. The base may be used in its solid form or an aqueous solution of a base may be used. Preferably, an aqueous solution of sodium hydroxide is used as a base for condensation. The condensation reaction is carried out in the absence of a phase-transfer catalyst.

The oxidizing agent used for the oxidation of sulphide intermediate of Formula VII may be selected from the group comprising of nitric acid, hydrogen peroxide, peracids such as peracetic acid, trifluoroperacetic acid, permaleic acid, m-chloroperbenzoic acid and the like, peresters, ozone, dinitrogentetraoxide, iodosobenzene, N-halosuccinimide, 1-chlorobenzotriazole, t-butylhypochlorite, diazobicyclo-[2,2,2]-octane bromine complex, sodium metaperiodate, selenium dioxide, manganese dioxide, chromic acid, cericammonium nitrate, bromine, chlorine, sulfuryl chloride, sodium bromite or sodium hypochlorite, magnesium monoperoxyphthalate, ammonium molybdate, vanadium oxide, iodosobenzene, methyliodosobenzene, sodium periodate and the like. Preferably, sodium hypochlorite is used for the oxidation of suphide of Formula VII to pantoprazole free base of Formula VIII. The oxidation reaction is carried out in the absence of a phase transfer catalyst.

The oxidizing agent may be added in the presence of a suitable solvent. The suitable solvent may be selected from the group comprising of alcohols, hydrocarbons, chlorinated hydrocarbons, ethers, alkyl acetates, ketones, dipolar aprotic solvents and/or mixtures thereof. Examples of alcohols may include straight and branched chain alcohols such as methanol, ethanol, n-propanol, iso-propanol and the like, cyclic alcohols such as cyclopentanol, cyclohexanol and the like, aromatic alcohols such as substituted or un-substituted benzyl alcohols and the like. Examples of hydrocarbons may include hexane, cyclohexane, benzene, toluene and the like. Examples of chlorinated hydrocarbons may include chloroform, dichloromethane and the like. Examples of ethers may include diethyl ether, diisopropyl ether, tetrahydrofuran and the like. Examples of alkyl acetates may include ethyl acetate, iso-propyl acetate and the like. Examples of ketones may include acetone, methyl ethyl ketone, methyl isobutyl ketone and the like. Examples of dipolar aprotic solvents may include acetonitrile, dimethylformamide, dimethylsulphoxide and the like. Preferably, the oxidizing agent is added in methanol.

A base may be added for carrying the oxidation reaction. The base may be selected from group of bases used for carrying out the condensation of 2-chloromethyl-3,4-dimethoxypyridine hydrochloride of Formula VI with 2-mercapto-5-difluoromethoxy benzimidazole of Formula V. The base may be added in its solid form or an aqueous solution of the base may be added.

The oxidizing agent may be added at a low temperature of about −35 to about 0° C. Preferably, oxidizing agent is added at a temperature of about −5 to −20° C.

Pantoprazole free base of Formula VIII can be converted to pantoprazole sodium of Formula IV by any of the processes described in the literature such as those described in PCT Publication No. WO 91/19710 and U.S. Publication No. 2005/0075370, which are herein incorporated by reference only. In general, pantoprazole free base may be converted to pantoprazole sodium by dissolving pantoprazole in acetone, adding an aqueous solution of sodium hydroxide and isolation. Isolation of the sodium salt may be facilitated by seeding.

Pantoprazole sodium obtained in the second and third aspect of the invention may be purified by any of the processes described in the literature. In general, the purification may be carried out by recrystallization from acetone.

Pantoprazole sodium obtained by the process described in the second and third aspect of the invention may be converted to hydrates and polymorphs of pantoprazole sodium. Examples of hydrates may include pantoprazole sesquihydrate, pantoprazole sodium trihydrate and the like.

The conversion of pantoprazole sodium obtained by the process described in the second and third aspect of the invention to pantoprazole sodium sesquihydrate may be carried out by the process described in the first aspect of the invention or by any of the processes described in the literature such as those described in U.S. Pat. Nos. 7,081,534 and 7,060,839, U.S. Publication No. 2004/0177804, PCT Publication No. WO 2007/017890 and J. Med. Chem., 1992, 35 (6), 1049, which are herein incorporated by reference only.

Pantoprazole sodium sesquihydrate obtained by the process of the invention can be converted to other hydrates and polymorphs of pantaprazole sodium by the processes described in the literature such as those described in PCT Publication No. WO 91/19710 and U.S. Publication No. 2004/0177804, which are herein incorporated by reference only.

Pantoprazole sodium of the present invention has a purity of about 99.9% by HPLC.

Pantoprazole sodium, obtained by the process of the present invention, is substantially free of sulphone impurities.

Pantoprazole sodium sesquihydrate, obtained by the process of the present invention is substantially free of sulphone impurities.

The term “substantially free of sulphone impurities” refers to pantoprazole sodium having no detectable amount of sulphone impurities.

In the foregoing section embodiments are described by way of examples to illustrate the process of invention. However, these are not intended in any way to limit the scope of the present invention. Several variants of these examples would be evident to persons ordinarily skilled in the art.

### Examples

### Example 1

### Preparation of Pantoprazole Sodium

2-Mercapto-5-difluoromethoxy benzimidazole (50 g) was added to an aqueous solution of sodium hydroxide (21.3 g in 350 mL de-ionized water) at room temperature to obtain a clear solution. An aqueous solution of 2-chloromethyl-3,4-dimethoxypyridine hydrochloride (50 g in 150 mL water) was added to the above solution over a period of about 2.0-2.5 hours. The reaction mixture was stirred vigorously for about 2-2.5 hours. Progress of the reaction was monitored by thin-layer chromatography. The reaction mixture was extracted with dichloromethane and washed with water. Organic layer was concentrated.

Methanol (50 mL) was added to the organic layer. The reaction mixture was cooled to −5 to −20° C. Aqueous solution of sodium hydroxide (11.8 g in 50 mL water) was added followed by addition of sodium hypochlorite solution (431 mL) in an aqueous solution of sodium hydroxide (20 g/100 mL) over a period of about 30 to about 45 minutes. The progress of the reaction was monitored by thin-layer chromatography. After completion of the reaction, the reaction mixture was quenched with 5% sodium hydrogen sulphite solution (500 mL). Water (500 mL) was added. pH of the reaction mixture was adjusted to 9.0-10.5. Layers were separated and the aqueous layer was extracted with dichloromethane. The combined dichloromethane layers were concentrated completely to obtain a red-brown colored residue.

The residue was dissolved in acetone (375 mL). The reaction mixture was cooled to 20-25° C. Aqueous solution of sodium hydroxide (9.2 g in 25 mL water) was added followed by addition of a seed crystal of pantoprazole sodium. The reaction mixture was stirred, cooled, stirred, filtered and washed with cold acetone to obtain crude pantoprazole sodium as a wet cake.

### Example 2

### Purification of Pantoprazole Sodium

The wet cake of pantoprazole sodium (115 g) obtained in example-1 was dissolved in acetone (250 mL) at reflux, charcoalized, filtered and washed with acetone to obtain a clear filtrate. The filtrate was partially concentrated under reduced pressure. The precipitated solid was stirred at room temperature for about 1 hour. The reaction mixture was cooled to about 10° C. to about 20° C., stirred for about 1 hour, filtered and washed with acetone to obtain pure pantoprazole sodium monohydrate.
Yield: 84 g
HPLC Purity: 99.9%

### Example 3

### Preparation of Pantoprazole Sodium Sesquihydrate

Pantoprazole sodium monohydrate (50 g) was dissolved in a mixture of dichloromethane (500 mL) and ethanol (30 mL). A seed crystal (0.2 g) was added followed by addition of dichloromethane (750 mL). The reaction mixture was cooled to about 0-5° C., stirred for about 5-6 hours, filtered and washed with dichloromethane to obtain pantoprazole sodium sesquihydrate.
Yield: 45 g
HPLC Purity: 99.9%
Water Content: 6.1-6.5

1. A process for the preparation of pantoprazole sodium sesquihydrate of Formula I 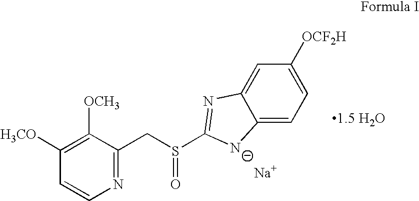
comprising
a) contacting a hydrate of pantoprazole sodium of Formula IV, which is not a sesquihydrate, 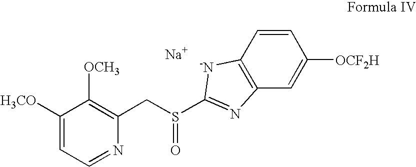
with a mixture of chlorinated solvent and alcohol, wherein the quantity of chlorinated solvent is more than that of alcohol,
b) optionally seeding the reaction mixture,
c) cooling the reaction mixture to a temperature of about −10° to about +10° C., and
d) isolating pantoprazole sodium sesquihydrate of Formula I from the mixture thereof.

2. The process according to claim 1, wherein chlorinated solvent is selected from the group consisting of chloroform, dichloromethane, and dichloroethane and mixtures thereof.

3. The process according to claim 1, wherein alcohol is selected from the group consisting of aliphatic alcohols such as methanol, ethanol, n-propanol, iso-propanol and butanol, alicyclic alcohols cyclopentanol and cyclohexanol.

4. (canceled)

5. The process according to claim 1, wherein obtained pantoprazole sodium sesquihydrate is substantially free of sulphone impurity.

6. A one-pot process for the preparation of pantoprazole sodium of Formula IV 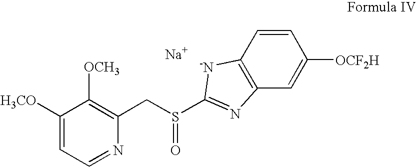
comprising condensation of 2-mercapto-5-difluoromethoxy benzimidazole of Formula V 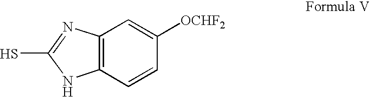
with 2-chloromethyl-3,4-dimethoxyptridine hydrochloride of Formula VI, 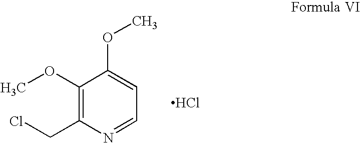
to obtain a sulphide intermediate of Formula VII, 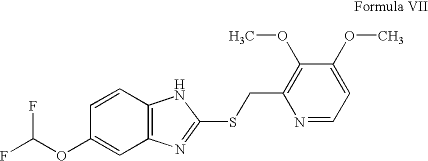
adding an oxidizing agent to obtain pantoprazole free base of Formula VIII 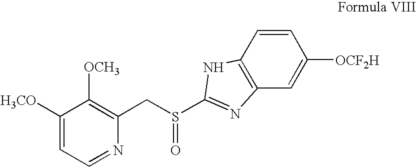
followed by its conversion to pantoprazole sodium, wherein the condensation and
oxidation reactions are carried out in the absence of a phase transfer catalyst.

7. The process according to claim 6, wherein condensation of 2-mercapto-5-difluoromethoxy benzimidazole of Formula V with 2-chloromethyl-3,4-dimethoxypyridine hydrochloride of Formula VI is carried out in the presence of a base.

8. The process according to claim 7, wherein the base is selected from the group consisting of inorganic bases such as hydroxides, carbonates, bicarbonates, acetates, alkoxides of alkali and alkaline earth metals.

9. The process according to claim 8, wherein the inorganic base is selected from the group consisting of sodium hydroxide, potassium hydroxide, sodium carbonate, potassium carbonate, potassium bicarbonate and mixtures thereof.

10. The process according to claim 9, wherein base is an aqueous solution of sodium hydroxide.

11. The process according to claim 6, wherein the oxidizing agent is selected from the group consisting of nitric acid, hydrogen peroxide, peracids such as peracetic acid, trifluoroperacetic acid, permaleic acid, m-chloroperbenzoic acid and the like, peresters, ozone, dinitrogentetraoxide, iodosobenzene, N-halosuccinimide, 1-chlorobenzotriazole, t-butylhypochlorite, diazobicyclo-[2,2,2]-octane bromine complex, sodium metaperiodate, selenium dioxide, manganese dioxide, chromic acid, cericammonium nitrate, bromine, chlorine, sulfuryl chloride, sodium bromite or sodium hypochlorite, magnesium monoperoxyphthalate, ammonium molybdate, vanadium oxide, iodosobenzene, methyliodosobenzene, and sodium periodate.

12. The process according to claim 6, wherein the oxidizing agent is added in the presence of a suitable solvent.

13. The process according to claim 12, wherein the suitable solvent is selected from the group consisting of alcohols, hydrocarbons, chlorinated hydrocarbons, ethers, alkyl acetates, ketones, dipolar aprotic solvents and/ or mixtures thereof.

14. The process according to claim 13, wherein alcohol is selected from the group consisting of aliphatic alcohols such as methanol, ethanol, n-propanol, iso-propanol and butanol, alicyclic alcohols cyclopentanol and cyclohexanol.

15. The process according to claim 6, wherein a base is added for carrying the oxidation reaction.

16. The process according to claim 15, wherein the base is selected from group of bases mentioned in claim 8.

17. The process according to claim 6, wherein the oxidizing agent is added at a low temperature of about −35 to about 0° C.

18. The process according to claim 6, wherein Pantoprazole sodium obtained is further converted to hydrates and polymorphs of pantoprazole sodium.

19. The process according to claim 1S, wherein Pantoprazole sodium obtained is further converted to pantoprazole sodium sesquihydrate.

20. The process according to claim 6, wherein obtained pantoprazole sodium is substantially free of sulphone impurity.

21. The process according to claim 6, wherein obtained Pantoprazole sodium has a purity of about 99.9% by HPLC.
